(19) United States
(12) Patent Application Publication
(10) Pub. No.: US 2006/0161169 A1
(43) Pub. Date: Jul. 20, 2006

(54) Device and method for modifying the shape of a body organ

(75) Inventors: Greg Nieminen, Bothell, WA (US); Mark L. Mathis, Fremont, CA (US); David Reuter, Bothell, WA (US)

Correspondence Address: WILSON SONSINI GOODRICH & ROSATI, 650 PAGE MILL ROAD, PALO ALTO, CA, 94304-1050, US

(73) Assignee: Cardiac Dimensions, Inc., a Delaware Corporation, Kirkland, WA (US)

(21) Appl. No.: 10/845,474

(22) Filed: May 12, 2004

Related U.S. Application Data
(63) Continuation-in-part of application No. 10/712,546, filed on Nov. 12, 2003.
Application No. 10/712,546 is a continuation-in-part of application No. 10/429,172, filed on May 2, 2003.
(60) Provisional application No. 60/476,695, filed on Jun. 5, 2003.

(51) Int. Cl. A61B 17/58

(52) U.S. Cl. 606/100

(57) ABSTRACT

The invention is a tissue shaping device. In some embodiments, the device includes an a reshaping element and an anchor adapted to anchor the device in a lumen, the anchor having a wire adapted to contact a wall of the lumen with an anchoring force when the device is deployed in the lumen and a force distribution element adapted to distribute the anchoring force more along a first anchoring axis than along a second anchoring axis. The invention is also a method of deploying a tissue shaping device in a lumen. In some embodiments the method includes the steps of placing an anchor in contact with a wall of the lumen to exert an anchoring force on the lumen wall; and distributing the anchoring force more along a first anchoring axis than along a second anchoring axis.

### CROSS-REFERENCE

This application is a continuation-in-part of U.S. patent application Ser. No. 10/712,546, “Tissue Shaping Device With Conformable Anchors,” filed Dec. 19, 2003, which is a continuation-in-part of U.S. patent application Ser. No. 10/429,172, “Device and Method for Modifying the Shape of a Body Organ,” filed May 2, 2003, both of which are incorporated herein by reference. This application also claims the benefit of U.S. Provisional Application No. 60/476,695, filed Jun. 5, 2003, which application is incorporated herein by reference.

### BACKGROUND OF THE INVENTION

The mitral valve is a portion of the heart that is located between the chambers of the left atrium and the left ventricle. When the left ventricle contracts to pump blood throughout the body, the mitral valve closes to prevent the blood from being pumped back into the left atrium. In some patients, whether due to genetic malformation, disease or injury, the mitral valve fails to close properly causing a condition known as regurgitation, whereby blood is pumped into the atrium upon each contraction of the heart muscle. Regurgitation is a serious, often rapidly deteriorating, condition that reduces circulatory efficiency and should be corrected.

Three of the more common techniques for restoring the function of a damaged mitral valve are to surgically replace the valve with a mechanical valve, to surgically repair the valve, or to suture a flexible ring around the valve to support it. Each of these procedures is highly invasive because access to the heart is obtained through an opening in the patient's chest. Patients with severe mitral valve regurgitation can be relatively frail thereby increasing the risks associated with such an operation.

One less invasive approach for aiding the closure of the mitral valve involves the placement of a support structure in the cardiac sinus and vessel that passes adjacent the mitral valve. The support structure is designed to push the vessel and surrounding tissue against the valve to aid its closure. This technique has the advantage over other methods of mitral valve repair because it can be performed percutaneously without opening the chest wall. Examples of such devices are shown in U.S. patent application Ser. No. 10/003,910, “Focused Compression Mitral Valve Device and Method;” U.S. patent application Ser. No. 10/142,637, “Body Lumen Device Anchor, Device and Assembly;” U.S. patent application Ser. No. 10/331,143, “System and Method to Effect the Mitral Valve Annulus of a Heart;” and U.S. patent application Ser. No. 10/429,172, “Device and Method for Modifying the Shape of a Body Organ,” filed May 2, 2003. The disclosures of these patent applications are incorporated herein by reference.

The purpose of a support device in a lumen such as a vein or artery is to reshape a particular tissue area adjacent to the lumen. In order to be minimally invasive, the reshaping should be limited to the target tissue, such as the mitral valve annulus, and any reshaping of other tissue adjacent to the lumen should be minimized or avoided. For example, to treat mitral valve regurgitation, the device is placed in the coronary sinus to reshape the mitral valve annulus. Care should be taken to minimize the reshaping of other adjacent tissue, such as nearby arteries. See, e.g., the following applications (the disclosures of which are incorporated herein by reference): U.S. patent application Ser. No. 09/855,945, “Mitral Valve Therapy Device, System and Method” (published Nov. 14, 2002, as US 2002/0169504 A1); U.S. patent application Ser. No. 09/855,946, “Mitral Valve Therapy Assembly and Method” (published Nov. 14, 2002, as US 2002/0169502 A1). It is also advisable to monitor cardiac perfusion during and after such mitral valve regurgitation therapy. See, e.g., U.S. patent application Ser. No. 10/366,585, “Method of Implanting a Mitral Valve Therapy Device,” the disclosure of which is incorporated herein by reference.

### SUMMARY OF THE INVENTION

The invention is a device for modifying the shape of tissue adjacent to a body lumen. One application for the device of this invention is in the treatment of mitral valve regurgitation.

One aspect of the invention is a tissue shaping device with a reshaping element and an anchor adapted to anchor the device in a lumen, the anchor having a wire adapted to contact a wall of the lumen with an anchoring force when the device is deployed in the lumen and one or more force distribution elements adapted to distribute the anchoring force more along a first anchoring axis than along a second anchoring axis, which may be substantially perpendicular to the first anchoring axis. In some embodiments, the wire is formed in a substantially figure 8 shape. In some embodiments, the force distribution element(s) may be a loop formed in the wire. The anchor may include a wire fastener, and the force distribution element may be configured so that the second anchoring axis passes through the wire fastener. The device may also have a second anchor adapted to anchor the device in the lumen, with the reshaping element extending between the two anchors.

Another aspect of the invention provides a method of deploying a tissue shaping device in a lumen, the tissue shaping device having an anchor and a reshaping element. The method may include the steps of placing the anchor in contact with a wall of the lumen to exert an anchoring force on the lumen wall; and distributing the anchoring force more along a first anchoring axis than along a second anchoring axis, such as by using at least one force distributor associated with (e.g., integral with) the anchor to distribute the anchoring force. In embodiments in which the anchor includes a wire, the placing step may include the step of placing the wire in contact with the lumen wall. In embodiments in which the anchor also includes a wire fastener, the distributing step may include the step of distributing the anchoring force more along an anchoring axis that does not pass through the wire fastener than along an anchoring axis that passes through the wire fastener. For example, the method may include the step of distributing the anchoring force more along an axis substantially perpendicular to the anchoring axis that passes through the wire fastener than along the axis passing through the wire fastener.

In some embodiments of the method, the placing step may include the step of exerting the anchoring force on the lumen wall substantially around an inner circumference of a section of the lumen. In aspect of these embodiments in which the anchor includes a wire and a wire fastener, the placing step may include the step of placing the wire and the wire fastener in contact with the lumen wall. The method may also include the step of placing a second anchor in contact with a wall of the lumen.

Other aspects of the invention will be apparent from the following detailed description and drawings.

### BRIEF DESCRIPTION OF THE DRAWINGS

FIG. 1 is an elevational view of an anchor for use with a tissue shaping device in accordance with one embodiment of the invention.

FIG. 2 is an end view along the line A-A of FIG. 1 of the anchor disposed in a lumen.

FIG. 3 is an elevational view of a tissue shaping device according to an embodiment of the invention.

### DETAILED DESCRIPTION OF THE INVENTION

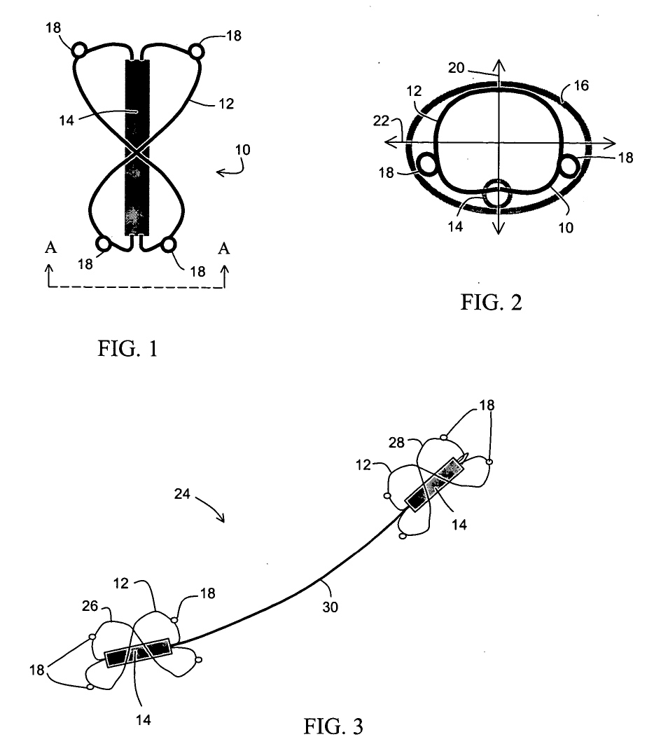

FIGS. 1 and 2 show an anchor design according to one embodiment of the invention. The anchor 10 of this invention includes one or more wires 12 formed in a figure 8 configuration held in place by a wire fastener, such as a crimp 14. wire 12 is preferably made of a shape memory material such as nitinol, and crimp 14 may be made of nitinol, titanium or some other suitable material. Anchor 10 is expandable within a body lumen from a collapsed delivery configuration to an expanded deployment configuration to anchor a tissue reshaping device within the lumen, such as the device shown in FIG. 3. Further details of the wire and crimp anchor design, the catheter-based delivery of such devices and of tissue reshaping in general may be found in U.S. patent application Ser. No. 10/142,637, “Body Lumen Device Anchor, Device and Assembly;” U.S. patent application Ser. No. 10/331,143, “System and Method to Effect the Mitral Valve Annulus of a Heart;” and U.S. patent application Ser. No. 10/429,172, “Device and Method for Modifying the Shape of a Body Organ,” filed May 2, 2003.

Anchor 10 performs its anchoring function by placing an outwardly directed force on the vessel wall 16 surrounding the lumen. As shown in FIG. 2, the anchor exerts its outwardly directed force around the entire circumference of the lumen wall within the section of lumen in which it is disposed. One or more loops 18 are formed in wire 12 to act as anchor force distribution elements. The way that loops 18 modify the distribution of the anchor's outwardly directed force depends on the size, shape and location of loops 18. In the embodiment shown in FIGS. 1-3, for example, the loops 18 reduce the anchor's outwardly directed force in the directions of arrows 20 with respect to the outwardly directed force in the directions of arrow 22, as seen best in FIG. 2 (which is a view of the anchor of FIG. 1 in the direction of A-A).

A device 24 such as that shown in FIG. 3 may be deployed in a coronary sinus to reshape the adjacent tissue of the mitral valve annulus to treat mitral valve regurgitation. Device 24 has two anchors 26 and 28 (formed as shown in FIGS. 1 and 2) connected by a reshaping element 30, such as one or more nitinol wires. One way that device 24 may be used to reshape the mitral valve annulus is by deploying anchor 26 in a distal location within the coronary sinus, cinching by pulling proximally on reshaping element 30, then deploying anchor 28 in a proximal location to maintain the cinched shape.

It may be desirable to minimize the outwardly directed force beneath and directly above crimps 14, such as to minimize the compression of any arteries beneath or directly above crimps 14. Loops 18 distribute the anchors' outwardly directed force so that less force is directed beneath and directly above crimps 14.

The anchor design of this invention may be used with other devices as well.

What is claimed is:

1. A tissue shaping device comprising a reshaping element and an anchor adapted to anchor the device in a lumen, the anchor comprising a wire adapted to contact a wall of the lumen with an anchoring force when the device is deployed in the lumen and a force distribution element adapted to distribute the anchoring force more along a first anchoring axis than along a second anchoring axis.

2. The tissue shaping device of claim 1 wherein the second anchoring axis is substantially perpendicular to the first anchoring axis.

3. The tissue shaping device of claim 1 wherein the anchor comprises a plurality of force distribution elements.

4. The tissue shaping device of claim 1 wherein the force distribution element comprises a loop formed in the wire.

5. The tissue shaping device of claim 1 wherein the wire is formed in a substantially figure 8 shape.

6. The tissue shaping device of claim 5 wherein the anchor comprises a plurality of force distribution elements.

7. The tissue shaping device of claim 6 wherein the force distribution elements each comprise a loop formed in the wire.

8. The tissue shaping device of claim 5 wherein the anchor further comprises a wire fastener.

9. The tissue shaping device of claim 8 wherein the force distribution element is configured so that the second anchoring axis passes through the wire fastener.

10. The tissue shaping device of claim 1 wherein the anchor is a first anchor, the device further comprising a second anchor adapted to anchor the device in the lumen, the reshaping element extending between the first and second anchors.

11. A method of deploying a tissue shaping device in a lumen, the tissue shaping element comprising an anchor and a reshaping element, the method comprising:
placing the anchor in contact with a wall of the lumen to exert an anchoring force on the lumen wall; and
distributing the anchoring force more along a first anchoring axis than along a second anchoring axis.

12. The method of claim 11 wherein the anchor comprises a wire, the placing step comprising placing the wire in contact with the lumen wall.

13. The method of claim 12 wherein the anchor further comprises a wire fastener, the distributing step comprising distributing the anchoring force more along an anchoring axis that does not pass through the wire fastener than along an anchoring axis that passes through the wire fastener.

14. The method of claim 12 wherein the anchor further comprises a wire fastener, the distributing step comprising distributing the anchoring force more along a first anchoring axis that does not pass through the wire fastener than along a second anchoring axis substantially perpendicular to the first anchoring axis that passes through the wire fastener.

15. The method of claim 11 wherein the placing step comprises exerting the anchoring force on the lumen wall substantially around an inner circumference of a section of the lumen.

16. The method of claim 15 wherein the anchor comprises a wire and a wire fastener, the placing step comprising placing the wire and the wire fastener in contact with the lumen wall.

17. The method of claim 11 wherein the second anchoring axis is substantially perpendicular to the first anchoring axis.

18. The method of claim 11 the step of distributing the anchoring force comprises using at least one force distributor associated with the anchor to distribute the anchoring force.

19. The method of claim 18 wherein the force distributor is integral with the anchor.

20. The method of claim 11 wherein the anchor is a first anchor, the method further comprising placing a second anchor in contact with a wall of the lumen.
